(19) United States
(12) Patent Application Publication
(10) Pub. No.: US 2025/0174317 A1
(43) Pub. Date: May 29, 2025

(54) METHOD FOR AUTOMATICALLY GENERATING DRAFT OF CLINICAL TRIAL DESIGN BASED ON LARGE LANGUAGE MODEL

(71) Applicant: MEDIAIPLUS CO., LTD., Seongnam-si (KR)

(72) Inventors: Nam Goo SONG, Suwon-si (KR); Ji Hee JUNG, Seoul (KR)

(21) Appl. No.: 18/956,723

(22) Filed: Nov. 22, 2024

(30) Foreign Application Priority Data
Nov. 29, 2023 (KR) 10-2023-0169139

(51) Int. Cl. G16H 10/20

(52) U.S. Cl. CPC G16H 10/20

(57) ABSTRACT

An embodiment relates to a method for automatically generating a draft of a clinical trial design based on a large language model (LLM), which is performed by a server, comprising: (a) inputting a plurality of pieces of clinical trial data to a predetermined LLM as training data and training the LLM; (b) receiving, from a user device, basic clinical trial information including a clinical trial title, a drug name, formulation, a target disease, and a phase of a clinical trial to be conducted; (c) combining a plurality of pieces of pre-stored query text with the basic clinical trial information to generate a plurality of pieces of final query text; and (d) inputting the plurality of pieces of final query text to the LLM to generate a clinical trial design draft report in which a plurality of response information strings is output for a plurality of sections, respectively.

### CROSS-REFERENCE TO RELATED APPLICATION

This application claims the benefit under 35 USC 119 (a) of Korean Patent Applications No. 10-2023-0169139 filed on Nov. 29, 2023 in the Korean Intellectual Property Office, the entire disclosures of which are incorporated herein by reference for all purposes.

### TECHNICAL FIELD

The present disclosure relates to a system for automatically generating a draft of a clinical trial design based on a large language model and a method for providing the same, and more particularly, to a method and system for automatically generating a draft of a clinical trial design by inputting clinical trial data to a large language model that has been trained with a plurality of pieces of information related to new drug development.

### BACKGROUND

Recently, as diseases induced by various mutant viruses have become rampant, the research and development of new drugs has been actively undertaken to cope with these diseases.

New drug development and research necessarily goes through clinical trials. In most cases, pharmaceutical companies conducting research and development of new drugs entrust clinical trials to Contract Research Organizations (CROs).

In this process, there are many difficulties in communication between the pharmaceutical companies and the CROs. A major reason is that clinical trials are in a specialized field different from new drug development and are conducted with various considerations for each clinical trial phase, each country and each drug, and, thus, it is essential to understand the latest regulations and guidelines, and the pharmaceutical companies cannot communicate with regulatory agencies responsible for clinical trial approval.

Accordingly, there is a growing need for a technology to easily and quickly generate a draft of a clinical trial design just with new drug information and basic clinical trial information, ensure smooth collaboration between pharmaceutical companies and CROs to conduct clinical trials for new drug development, and reduce the time and cost involved in new drug development.

### SUMMARY

In view of the foregoing, the present disclosure is conceived to provide a system for automatically generating a draft of a clinical trial design based on a large language model.

However, the problems to be solved by the present disclosure are not limited to the above-described problems. Although not described herein, other problems to be solved by the present disclosure can be clearly understood by a person with ordinary skill in the art from the following descriptions.

An aspect of the present disclosure provides a method for automatically generating a draft of a clinical trial design based on a large language model (LLM), which is performed by a server, including: (a) a process of inputting a plurality of pieces of clinical trial data to a predetermined LLM as training data and training the LLM; (b) a process of receiving, from a user device, basic clinical trial information including a clinical trial title, a drug name, formulation, a target disease, and a phase of a clinical trial to be conducted; (c) a process of combining a plurality of pieces of pre-stored query text with the basic clinical trial information to generate a plurality of pieces of final query text; and (d) a process of inputting the plurality of pieces of final query text to the LLM to generate a clinical trial design draft report in which a plurality of response information strings is output for a plurality of sections, respectively.

Also, the process (a) includes: (a-1) a process of receiving the plurality of pieces of clinical trial data from the user device; (a-2) a process of extracting a query information string including a clinical trial title, a disease name, and a clinical trial phase and a response information string including clinical trial patient recruitment criteria and clinical trial patient group design information from the received clinical trial data, and generating a prompt command by using the pre-stored query text, the query information string, and the response information string; and (a-3) a process of matching the prompt command with the query information string and the response information string to create a training dataset and adjusting parameters of the LLM by training the LLM with the training dataset.

Further, when the query text and the extracted query information string are input to the LLM, the LLM is trained to output the response information string for each of the sections, and the sections include a clinical trial abstract, a clinical trial setting, a clinical trial method, a patient group formation, drug information, and a clinical trial objective.

Furthermore, information in each of the sections is extracted from the LLM based on most frequently occurring keywords in the clinical trial report by training the LLM with the plurality of pieces of clinical trial data.

Moreover, the plurality of pieces of clinical trial data includes report files, image files, and video files of a plurality of clinical trials input from the user device or collected via internet crawling.

Also, the process (a) further includes: (a-4) a process of performing validation by setting clinical trial data for validation as a separate validation dataset among the plurality of pieces of clinical trial data set by a user, training the LLM with the validation dataset, and adjusting the parameters of the LLM.

Further, the final query text includes query text and a basic clinical trial information string, and the query text is located before the basic clinical trial information string.

The query text consists of a query or command to make a draft for a specific purpose by using the basic clinical trial information string, which sequentially lists information input by the user.

Furthermore, the process (c) further includes: (c-1) a process of analyzing a meaning of a string constituting the basic clinical trial information input from the user device and distinguishing strings corresponding to the title, the drug name, the formulation, the target disease, and the phase of the clinical trial, respectively, from the string constituting the basic clinical trial information.

Moreover, the process (c-1) further includes: a process of identifying a category of missing essential information constituting the basic clinical trial information from the string input from the user device when it is determined that the essential information is missed and requesting input of the essential information from the user device.

Also, the process (c-1) further includes: a process of analyzing the meaning of the string constituting the basic clinical trial information even when the user device inputs a plurality of strings without spaces, distinguishing the strings corresponding to the title, the drug name, the formulation, the target disease, and the clinical trial phase, respectively, from the string constituting the basic clinical trial information, and providing the user device with the strings with line spacing to distinguish categories of the title, the drug name, the formulation, the target disease, and the clinical trial phase, respectively, to query again whether it has been intended by the user.

Further, the process (d) further includes: a process of reconstructing final query text when the number of response strings received is smaller than a predetermined threshold value after the final query text is input, and replacing a word in the final query text with another word or generating a prompt command in which a string constituting the basic clinical trial information is combined with query text into one sentence before inputting it to the LLM.

Furthermore, in the process (d), response information strings are sequentially generated for the plurality of sections, respectively, and when any one of the response information strings is generated, the response information string as well as query text and basic clinical trial information corresponding to the response information string are stored, and then, a response information string for a next section is generated.

Moreover, the method further includes: (e) a process of providing the clinical trial design draft report in a program format that allows for editing and saving on a webpage or application.

Also, in the process (e), keywords including a clinical trial method, a patient group, and drug information in the clinical trial design draft report are automatically generated in bold or highlighted.

Another aspect of the present disclosure provides a server for automatically generating a draft of a clinical trial design based on an LLM, including: a memory that stores a program configured to perform a method for automatically generating the draft of the clinical trial design based on the LLM; and a processor that executes the program, and the method includes: (a) a process of inputting a plurality of pieces of clinical trial data to a predetermined LLM as training data and training the LLM; (b) a process of receiving, from a user device, basic clinical trial information including a clinical trial title, a drug name, formulation, a target disease, and a phase of a clinical trial to be conducted; (c) a process of combining a plurality of pieces of pre-stored query text with the basic clinical trial information to generate a plurality of pieces of final query text; and (d) a process of inputting the plurality of pieces of final query text to the LLM to generate a clinical trial design draft report in which a plurality of response information strings is output for a plurality of sections, respectively.

According to an embodiment of the present disclosure, a system for automatically generating a draft of a clinical trial design based on an LLM makes it possible to easily and quickly generate a draft of a clinical trial design just with new drug information and basic clinical trial information.

Also, it is possible to ensure smooth collaboration between pharmaceutical companies and CROs to conduct clinical trials for new drug development and reduce the time and cost involved in new drug development. Therefore, the time and cost conventionally required can be invested in other development processes, which allows for faster new drug development.

### BRIEF DESCRIPTION OF THE DRAWINGS

In the detailed description that follows, embodiments are described as illustrations only since various changes and modifications will become apparent to a person with ordinary skill in the art from the following detailed description. The use of the same reference numbers in different FIG.s indicates similar or identical items.

FIG. 1 is a structure diagram of a system for automatically generating a draft of a clinical trial design based on an LLM according to an embodiment of the present disclosure.

FIG. 2 is a block diagram showing an internal configuration of a server according to an embodiment of the present disclosure.

FIG. 3 is a block diagram showing a configuration and operations of the system for automatically generating a draft of a clinical trial design based on an LLM according to an embodiment of the present disclosure.

FIG. 4 shows an example of query text and a query information string according to an embodiment of the present disclosure.

FIG. 5 shows an example of a clinical trial design draft report according to an embodiment of the present disclosure.

FIG. 6 is a flowchart showing a method for automatically generating a draft of a clinical trial design based on an LLM according to an embodiment of the present disclosure.

### DETAILED DESCRIPTION

Hereafter, embodiments will be described in detail with reference to the accompanying drawings so that the present disclosure may be readily implemented by a person with ordinary skill in the art. However, it is to be noted that the present disclosure is not limited to the embodiments but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected to” another element and an element being “electronically connected to” another element via another element. Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Throughout the whole document, the term “unit” includes a unit implemented by hardware, a unit implemented by software, and a unit implemented by both of them. One unit may be implemented by two or more pieces of hardware, and two or more units may be implemented by one piece of hardware. Meanwhile, the units are not limited to the software or the hardware, and each of the units may be stored in an addressable storage medium or may be configured to implement one or more processors. Accordingly, the units may include, for example, software, object-oriented software, classes, tasks, processes, functions, attributes, procedures, sub-routines, segments of program codes, drivers, firmware, micro codes, circuits, data, database, data structures, tables, arrays, variables and the like. The components and the functions of the units can be combined with each other or can be divided up into additional components and units. Further, the components and the “units” may be configured to implement one or more CPUs in a device or a secure multimedia card.

The term “device” to be described below may be implemented with computers or portable devices which can access a server or another device through a network. Herein, the computers may include, for example, a notebook, a desktop, a laptop, and a VR HMD (e.g., HTC VIVE, Oculus Rift, GearVR, DayDream, PSVR, etc.) equipped with a WEB browser. Herein, the VR HMD includes all of models for PC (e.g., HTC VIVE, Oculus Rift, FOVE, Deepon, etc.), mobile (e.g., GearVR, DayDream, Baofeng Mojing, Google Cardboard, etc.) and console (PSVR), and stand-alone models (e.g., Deepon, PICO, etc.). The portable devices are, for example, wireless communication devices that ensure portability and mobility and may include a smart phone, a tablet PC, a wearable device and various kinds of devices equipped with a communication module such as Bluetooth (BLE, Bluetooth Low Energy), NFC, RFID, ultrasonic waves, infrared rays, WiFi, LiFi, and the like. Further, the term “network” refers to a connection structure that enables information exchange between nodes such as devices, servers, etc. and includes LAN (Local Area Network), WAN (Wide Area Network), Internet (WWW: World Wide Web), a wired or wireless data communication network, a telecommunication network, a wired or wireless television network, and the like. Examples of the wireless data communication network may include 3G, 4G, 5G, 3GPP (3rd Generation Partnership Project), LTE (Long Term Evolution), WIMAX (World Interoperability for Microwave Access), Wi-Fi, Bluetooth communication, infrared communication, ultrasonic communication, VLC (Visible Light Communication), LiFi, and the like, but may not be limited thereto.

The present disclosure relates to a system for automatically generating a draft of a clinical trial design based on an LLM and a method for providing the same, and more particularly to, a technology for automatically generating a draft of a clinical trial design by inputting clinical trial data to an LLM that has been trained with a plurality of pieces of information related to new drug development.

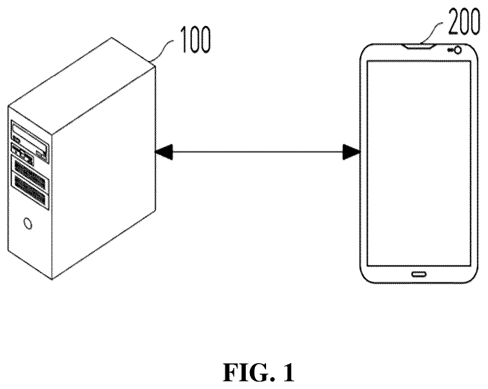

Referring to FIG. 1, the system for automatically generating a draft of a clinical trial design based on an LLM according to an embodiment of the present disclosure is composed of a server 100 and a user device 200.

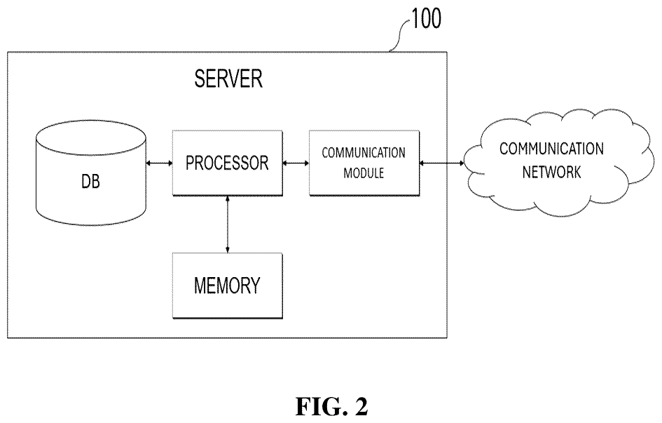

As shown in FIG. 2, the server 100 according to an embodiment of the present disclosure may be composed of a memory that stores a program (or application) configured to perform the method for automatically generating a draft of a clinical trial design based on an LLM, a processor that executes the program, and a data base (DB) that stores data for executing the program.

Also, the server 100 includes an embedded communication module which is connected to a communication network wired or wirelessly for communication, and the processor may perform various functions as the program stored in the memory is executed. The execution and functions of the program by the processor and the memory will be described in detail below.

In the method for automatically generating a draft of a clinical trial design based on an LLM according to an embodiment of the present disclosure, which is performed by the server 100, the server 100 inputs a plurality of pieces of clinical trial data to a predetermined LLM as training data and trains the LLM.

To this end, the server 100 receives the plurality of pieces of clinical trial data from the user device 200, extracts a query information string 120 including a clinical trial title, a disease name, and a clinical trial phase and a response information string including clinical trial patient recruitment criteria and clinical trial patient group design information from the received clinical trial data, and generates a prompt command by using query text 110 previously stored in the DB or the memory, the query information string 120, and the response information string.

In this case, the plurality of pieces of clinical trial data includes report files, image files, and video files of a plurality of clinical trials input to the server 100 from the user device 200 or collected by the server 100 via internet crawling. When these files are input from the user device 200, they may include confidential files and documents. When these files are collected via internet crawling, they may include documents and files publicly available through specific internet pages.

Also, the server 100 matches the generated prompt command with the query information string 120 and the response information string to create a training dataset and adjusts parameters of the LLM by training the LLM with the training dataset.

Herein, the LLM of the present disclosure is trained to output response information strings for respective sections 210 when the query text 110 and the extracted query information string 120 are input. According to various embodiments of the present disclosure, various LLMs can be used, such as OpenAI's ChatGPT or GPT-3, Google's Bard or Turing NLG, and META's LaMDA or Llama.

The sections 210 in which the LLM outputs the response information strings may include a include a clinical trial abstract, a clinical trial setting, a clinical trial method, a patient group formation, drug information, and a clinical trial objective, and information in each of the sections 210 is extracted from the LLM based on most frequently occurring keywords in a clinical trial report to be described below by training the LLM with the plurality of pieces of clinical trial data.

Through the above-described processes, the server 100 according to an embodiment of the present disclosure can train the LLM with the plurality of pieces of clinical trial data.

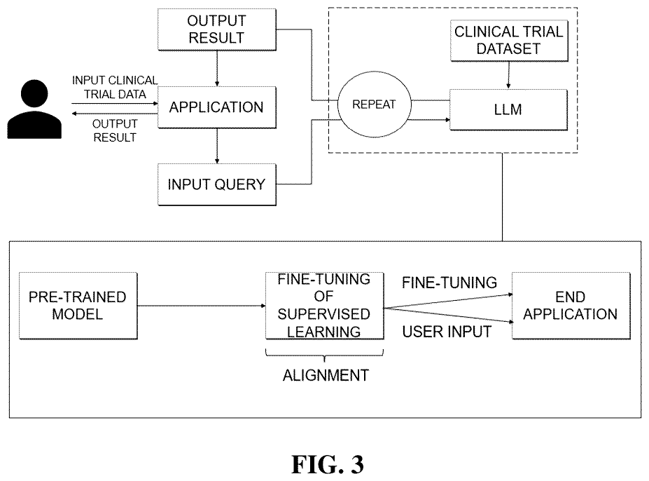

Referring to FIG. 3, the LLM may be trained repeatedly, and, thus, the server 100 performs supervised fine-tuning (alignment) on a pre-trained model to prepare a final downstream application or final program for use in subsequent processes to be described below.

Further, according to another embodiment of the present disclosure, the server 100 may perform validation by setting clinical trial data for validation as a separate validation dataset among the plurality of pieces of clinical trial data set by a user, i.e., clinical trial data input from the user device 200, training the LLM with the validation dataset, and further adjusting the parameters of the LLM.

Therefore, the LLM of the present disclosure does not use a large language model disclosed in the prior art, but rather involves performing fine-tuning for the efficient use of the LLM. Accordingly, the LLM of the present disclosure is trained and controlled to derive superior results compared to general large language models.

After the above-described processes, the server 100 receives, from the user device 200, basic clinical trial information including a clinical trial title, a drug name, formulation, a target disease, and a phase of a clinical trial to be conducted.

In the present disclosure, clinical trials include both animal and human experiments, and refer to a broad range of trials encompassing clinical pharmacology trials, therapeutic exploratory clinical trials, therapeutic confirmatory clinical trials, and therapeutic use clinical trials. The clinical trials described below shall be taken to include the above-described types of clinical trials.

Then, the server 100 combines a plurality of pieces of pre-stored query text 110 with the basic clinical trial information to generate a plurality of pieces of final query text.

Herein, the query text 110 consists of a query or command to make a draft for a specific purpose by using the basic clinical trial information string. The basic clinical trial information string sequentially lists information input by the user and may be previously stored in the DB or the memory of the server 100.

Further, the final query text includes the query text 110 and the basic clinical trial information string, and the query text 110 may be located before the basic clinical trial information string.

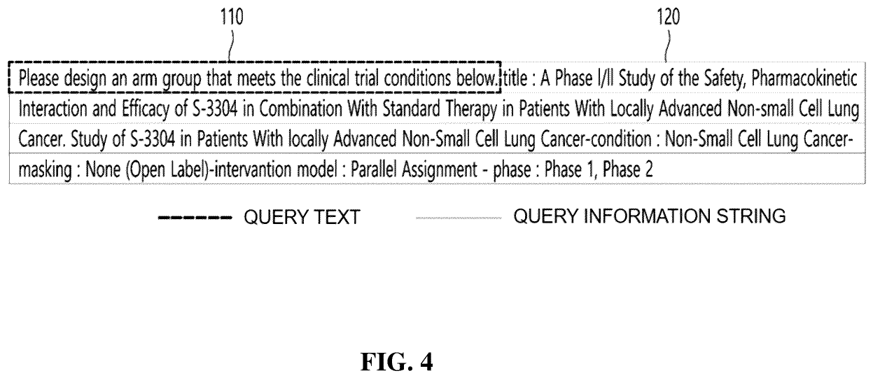

Referring to FIG. 4, the query text 110 and query information string 120 are described sequentially in the final query text according to an embodiment of the present disclosure, and the query text 110 may be generated to be located before query information string 120.

This is to maximize the efficiency of the LLM. Considering that most of the developers of large language models are based in English-speaking countries, it is described as a head-first sentence (main point, i.e., command, first, followed by detailed conditions) according to the English word order.

To generate the final query text, the server 100 analyzes a meaning of a string constituting the basic clinical trial information input from the user device 200 and distinguishes strings corresponding to the title, the drug name, the formulation, the target disease, and the phase of the clinical trial, respectively, from the string constituting the basic clinical trial information.

Herein, the server 100 identifies a category of missing essential information constituting the basic clinical trial information from the string input from the user device 200 when it is determined that the essential information is missed and requesting input of the essential information from the user device 200.

Also, the server 100 analyzes the meaning of the string constituting the basic clinical trial information even when the user device 200 inputs a plurality of strings without spaces, distinguishes the strings corresponding to the title, the drug name, the formulation, the target disease, and the clinical trial phase, respectively, from the string constituting the basic clinical trial information, and provides the user device 200 with the strings with line spacing to distinguish categories of the title, the drug name, the formulation, the target disease, and the clinical trial phase, respectively, to query again whether the current input value has been intended by the user.

Then, the server 100 inputs the final query text generated through the above-described processes to the LLM to generate a clinical trial design draft report in which a plurality of response information strings is output for a plurality of sections 210, respectively.

Herein, the server 100 reconstructs final query text when the number of response strings received is smaller than a predetermined threshold value after the final query text is input, and replaces a word in the final query text with another word or generating a prompt command in which a string constituting the basic clinical trial information is combined with the query text 110 into one sentence before inputting it to the LLM.

Due to the nature of large language models, which are trained to output responses as similar as possible to those of humans, even when queries essentially mean the same thing, different responses may be output depending on the word order and the types of words used. Therefore, according to the present disclosure, the queries can be repeated without limitation until a user-specified threshold value, i.e., a report length, is met through the above-described processes. Accordingly, it is possible to output superior results compared to conventional large language models.

Meanwhile, the LLM sequentially generates response information strings for the plurality of sections 210, respectively, in response to the final query text and when any one of the response information strings is generated, the response information string as well as the query text 110 and basic clinical trial information corresponding to the response information string are stored, and then, a response information string for a next section 210 is generated.

According to another embodiment of the present disclosure, the response information strings stored for the respective sections 210 can be provided to the user device 200 as a plurality of responses, such as Option 1, Option 2, and Option N, and the report can be edited by including a response selected by the user device 200.

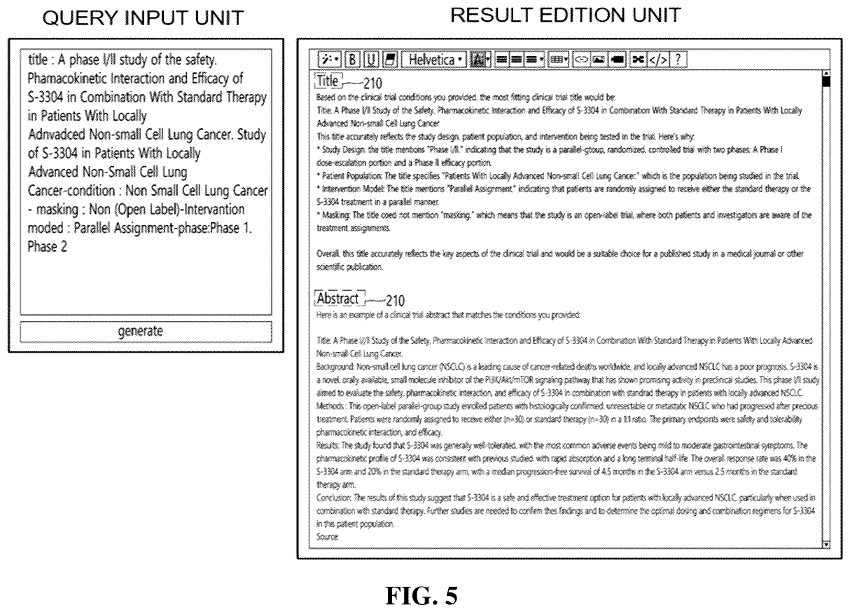

Referring to FIG. 5, the server 100 makes a clinical trial design draft report with the text in which the generated response information strings are output for the respective sections 210 and provides it in a program format that allows for editing and saving on a webpage or application.

Herein, keywords including a clinical trial method, a patient group, and drug information or the sections 210 in the clinical trial design draft report are automatically generated in bold or highlighted. If the report is edited by the user device 200 in the program that allows for editing and saving, the server 100 may compare the provided draft with the edited version and perform additional training with the difference therebetween as a part of the dataset.

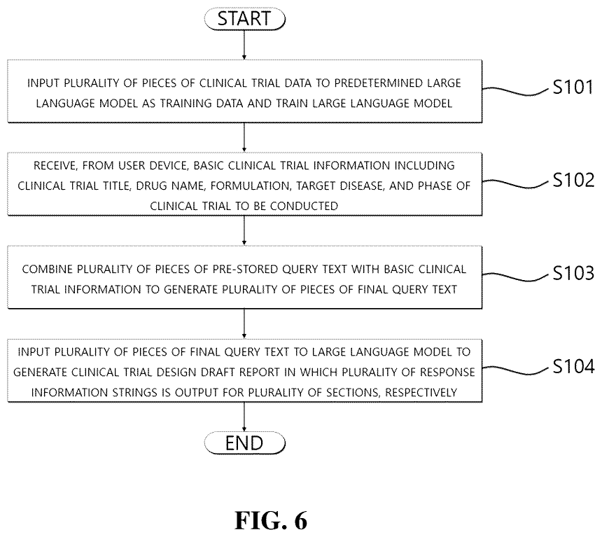

Hereafter, the execution sequence of the method for automatically generating a draft of a clinical trial design based on an LLM according to an embodiment of the present disclosure will be described again with reference to FIG. 6.

First, the server 100 inputs a plurality of pieces of clinical trial data to a predetermined LLM as training data and trains the LLM (S101).

Also, the server 100 receives, from the user device 200, basic clinical trial information including a clinical trial title, a drug name, formulation, a target disease, and a phase of a clinical trial to be conducted (S102).

Then, the server 100 combines the plurality of pieces of pre-stored query text 110 with the basic clinical trial information to generate a plurality of pieces of final query text (S103).

Thereafter, the server 100 inputs the plurality of pieces of final query text to the LLM to generate a clinical trial design draft report in which a plurality of response information strings is output for the plurality of sections 210, respectively (S104).

The embodiment of the present disclosure can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information such as computer-readable instruction code, a data structure, a program module or other data.

The method and system of the present disclosure have been explained in relation to a specific embodiment, but their components or a part or all of their operations can be embodied by using a computer system having general-purpose hardware architecture.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### EXPLANATION OF CODES

100: Server
110: Query text
120: Query information string
200: User device
210: Section

What is claimed is:

1. A method for automatically generating a draft of a clinical trial design based on a large language model (LLM), which is performed by a server, comprising:
(a) inputting a plurality of pieces of clinical trial data to a predetermined LLM as training data and training the LLM;
(b) receiving, from a user device, basic clinical trial information including a clinical trial title, a drug name, formulation, a target disease, and a phase of a clinical trial to be conducted;
(c) combining a plurality of pieces of pre-stored query text with the basic clinical trial information to generate a plurality of pieces of final query text; and
(d) inputting the plurality of pieces of final query text to the LLM to generate a clinical trial design draft report in which a plurality of response information strings is output for a plurality of sections, respectively.

2. The method of claim 1,
wherein the process (a) comprises:
(a-1) receiving the plurality of pieces of clinical trial data from the user device;
(a-2) extracting a query information string including a clinical trial title, a disease name, and a clinical trial phase and a response information string including clinical trial patient recruitment criteria and clinical trial patient group design information from the received clinical trial data, and generating a prompt command by using the pre-stored query text, the query information string, and the response information string; and
(a-3) matching the prompt command with the query information string and the response information string to create a training dataset and adjusting parameters of the LLM by training the LLM with the training dataset.

3. The method of claim 2,
wherein when the query text and the extracted query information string are input to the LLM, the LLM is trained to output the response information string for each of the sections, and
the sections include a clinical trial abstract, a clinical trial setting, a clinical trial method, a patient group formation, drug information, and a clinical trial objective.

4. The method of claim 3,
wherein information in each of the sections is extracted from the LLM based on most frequently occurring keywords in the clinical trial report by training the LLM with the plurality of pieces of clinical trial data.

5. The method of claim 2,
wherein the plurality of pieces of clinical trial data includes report files, image files, and video files of a plurality of clinical trials input from the user device or collected via internet crawling.

6. The method of claim 2,
wherein the process (a) further comprises:
(a-4) performing validation by setting clinical trial data for validation as a separate validation dataset among the plurality of pieces of clinical trial data set by a user, training the LLM with the validation dataset, and adjusting the parameters of the LLM.

7. The method of claim 1,
wherein the final query text includes query text and a basic clinical trial information string, and the query text is located before the basic clinical trial information string.

8. The method of claim 7,
wherein the query text consists of a query or command to make a draft for a specific purpose by using the basic clinical trial information string, which sequentially lists information input by a user.

9. The method of claim 1,
wherein the process (c) further comprises:
(c-1) analyzing a meaning of a string constituting the basic clinical trial information input from the user device and distinguishing strings corresponding to the title, the drug name, the formulation, the target disease, and the clinical trial phase, respectively, from the string constituting the basic clinical trial information.

10. The method of claim 9,
wherein the process (c-1) further comprises:
identifying a category of missing essential information constituting the basic clinical trial information from the string input from the user device when it is determined that the essential information is missed and requesting input of the essential information from the user device.

11. The method of claim 10,
wherein the process (c-1) further comprises:
analyzing the meaning of the string constituting the basic clinical trial information even when the user device inputs a plurality of strings without spaces, distinguishing the strings corresponding to the title, the drug name, the formulation, the target disease, and the clinical trial phase, respectively, from the string constituting the basic clinical trial information, and providing the user device with the strings with line spacing to distinguish categories of the title, the drug name, the formulation, the target disease, and the clinical trial phase, respectively, to query again whether it has been intended by a user.

12. The method of claim 1,
wherein the process (d) further comprises:
reconstructing final query text when the number of response strings received is smaller than a predetermined threshold value after the final query text is input, and replacing a word in the final query text with another word or generating a prompt command in which a string constituting the basic clinical trial information is combined with query text into one sentence before inputting it to the LLM.

13. The method of claim 1,
wherein in the process (d), response information strings are sequentially generated for the plurality of sections, respectively, and when any one of the response information strings is generated, the response information string as well as query text and basic clinical trial information corresponding to the response information string are stored, and then, a response information string for a next section is generated.

14. The method of claim 1, further comprising:
(e) providing the clinical trial design draft report in a program format that allows for editing and saving on a webpage or application.

15. The method of claim 13,
wherein in the process (e), keywords including a clinical trial method, a patient group, and drug information in the clinical trial design draft report are automatically generated in bold or highlighted.

16. A server for automatically generating a draft of a clinical trial design based on an LLM, comprising:
a memory that stores a program configured to perform a method for automatically generating the draft of the clinical trial design based on the LLM; and
a processor that executes the program,
wherein the method includes:
(a) inputting a plurality of pieces of clinical trial data to a predetermined LLM as training data and training the LLM;
(b) receiving, from a user device, basic clinical trial information including a clinical trial title, a drug name, formulation, a target disease, and a phase of a clinical trial to be conducted;
(c) combining a plurality of pieces of pre-stored query text with the basic clinical trial information to generate a plurality of pieces of final query text; and
(d) inputting the plurality of pieces of final query text to the LLM to generate a clinical trial design draft report in which a plurality of response information strings is output for a plurality of sections, respectively.
